(19) United States
(12) Patent Application Publication
(10) Pub. No.: US 2012/0101518 A1
(43) Pub. Date: Apr. 26, 2012

(54) DePond clamp

(75) Inventor: Robert Todd DePond, Charleston, WV (US)

(73) Assignee: West Virginia University

(21) Appl. No.: 12/925,575

(22) Filed: Oct. 25, 2010

(51) Int. Cl. A61B 17/28

(52) U.S. Cl. 606/205

(57) ABSTRACT

A first embodiment can be a clamp for gripping viscera comprising a pair of arms, each arm including a proximal handle and a distal tissue engaging member, pivot means interconnecting said arms, and a locking means carried by each arm interengaging when said members are in clamping engagement, and one member extended distally with a bulbous tip.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BRIEF DESCRIPTION OF THE SEVERAL VIEWS OF THE DRAWING

FIG. 1 shows an embodiment of the clamp in a nearly fully engaged position.

FIG. 2 shows an embodiment of the clamp viewed on edge.

FIG. 3 show a fragmentary view of an embodiment of the clamp on an increased scale displaying the pivot, tissue engaging members, and distal ends of the arms.

FIG. 4 shows a fragmentary view of a curved embodiment of the clamp on an increased scale displaying the pivot, tissue engaging members, and distal ends of the arms.

FIG. 5 shows a curved embodiment of the clamp viewed on edge.

### DETAILED DESCRIPTION OF THE INVENTION

A first embodiment may be a scissors-like clamping device comprising two resilient arms 1 and 2 pivotally inter-connected between their ends, as at 12, in a conventional manner. The arms 1 and 2 have handles 3 and 4, respectively, at the proximal ends. Adjacent the handle 3, the arm 1 has an offset portion 5 provided with a series of transverse locking ridges 6. Adjacent the handle 4, the arm 1 has an offset portion 7 disposed to underlie the portion 5 when the clamp is closed and provided with a series of transverse locking ridges 8 disposed so that, when the clamp is attached, and depending on the clamping pressure exerted, one or more of the two series of ridges interengage to lock the clamp. The interengaged ridges are readily disengaged by slight, relative sideways movement of the handles. The distal ends of the arms 1 and 2 are provided with tissue engaging members 9 and 10, respectively. Said tissue engaging members are disposed towards each other. The clamp as thus far described is conventional.

In accordance with the invention, the engaging member 10 of the distal end of arm 2 is further distally provided with a non-engaging bulbous tip 11.

The bulbous tip is any shape on the end which provides a smooth surface for the distal end of the clamp when engaged. The bulbous tip thus allows the distal portion of the engaged clamp to slide along surfaces with less risk than a conventional clamp of catching or tearing tissues.

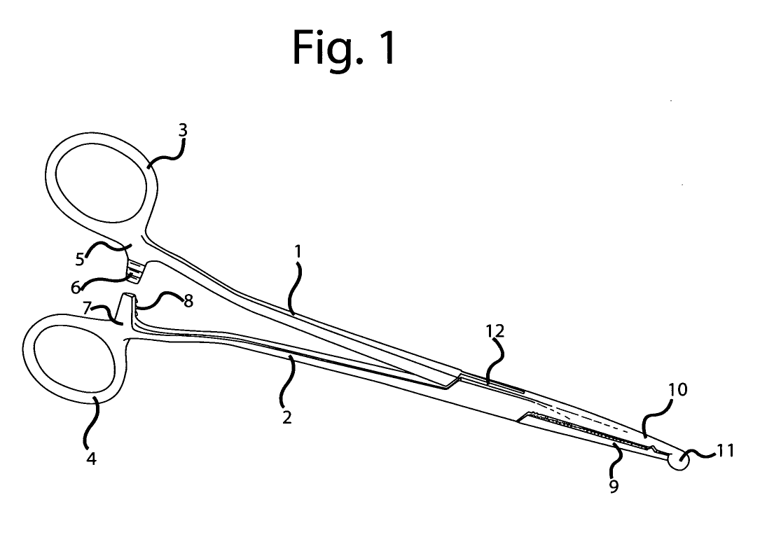

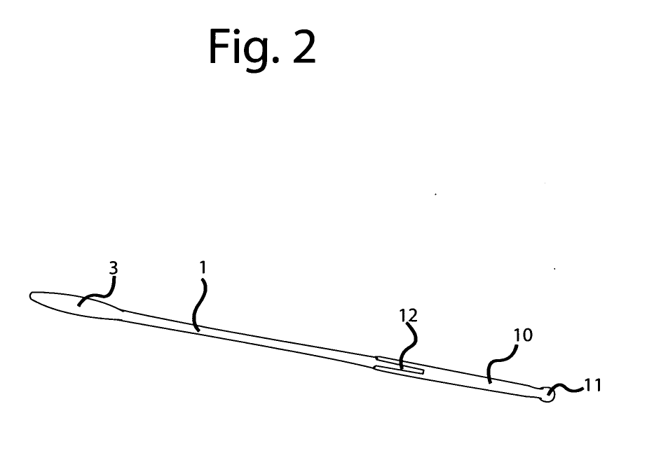

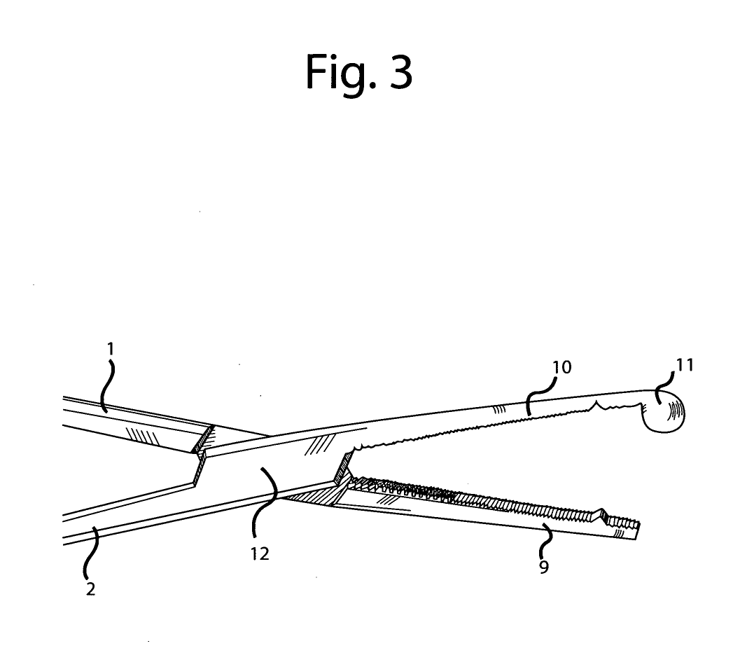

In FIGS. 1, 2, and 3 there is shown an embodiment with elongated tissue engaging members. The tissue engaging members on each arm have varied textures on the tissue engaging surface which correlate to similar areas on the corresponding tissue engaging surface of the inter-connected arm as can be seen in FIG. 3. The bulbous tip 11 of arm 2 protrudes such that it covers the distal tip of arm 1 thereby preventing the non-bulbous distal tip of arm 1 from contact with tissue when the clamp is in the engaged position. The distal surface of the engaged clamp is therefore the smooth bulbous form 11 at the distal end of arm 2.

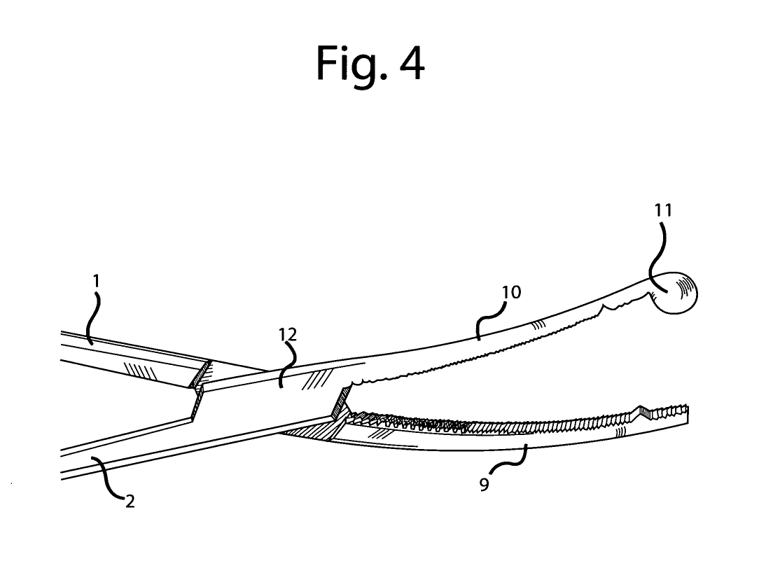

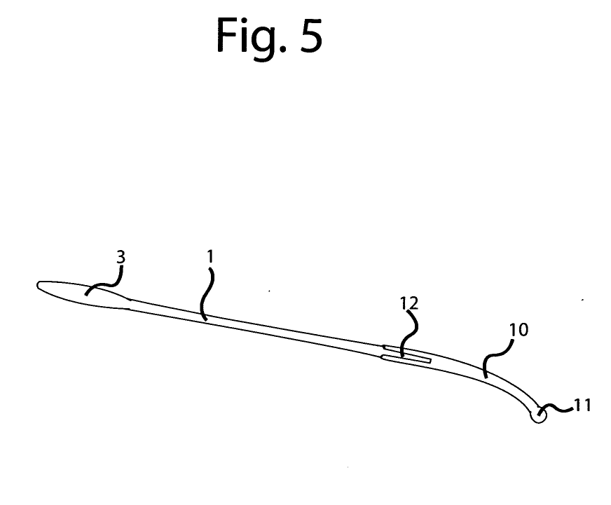

FIG. 4 depicts another embodiment with the tissue engaging portions 9 and 10 both curved rather than straight. This curve may be in one or more arcs and in one or more axes. FIG. 5 depicts an alternatively curved embodiment of the clamp as viewed from the edge.

What is claimed is:

1. A clamp for gripping viscera comprising a pair of arms, each arm including a proximal handle and a distal tissue engaging member, pivot means interconnecting said arms, and a locking means carried by each arm interengaging when said members are in clamping engagement, and one member extended distally with a bulbous tip.

2. The clamp for gripping viscera of claim 1 wherein the distal tissue engaging members of each arm are curved.
